(19) United States
(12) Patent Application Publication
(10) Pub. No.: US 2007/0264215 A1
(43) Pub. Date: Nov. 15, 2007

(54) Assortment of antiperspirants having two or more product performance characteristics

(76) Inventors: Timothy Alan Scavone, Loveland, OH (US); Theresa Louise Johnson, South Lebanon, OH (US); Michael Edward Thomas, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/598,405

(22) Filed: Nov. 13, 2006

Related U.S. Application Data
(63) Continuation-in-part of application No. 11/451,702, filed on Jun. 13, 2006.
Application No. 11/451,702 is a continuation-in-part of application No. 11/150,980, filed on Jun. 13, 2005.

(51) Int. Cl. G09B 25/00; A61K 8/00

(52) U.S. Cl. 424/65; 434/367

(57) ABSTRACT

An array of antiperspirant products including a first antiperspirant product sold under a first brand name, a second antiperspirant product also sold under the first brand name, wherein the second antiperspirant product is marketed as clinical strength or prescription strength and is substantially devoid of an antiperspirant active comprising aluminum chloride.

### CROSS REFERENCE TO RELATED APPLICATION

This application is a continuation-in-part of U.S. application Ser. No. 11/451,702, filed on Jun. 13, 2006, which is a continuation-in-part of U.S. application Ser. No. 11/150,980, filed Jun. 13, 2005.

### FIELD OF THE INVENTION

The present invention is related to a line of consumer products, particularly, antiperspirants having two or more product performance characteristics comprising a sequence of designs and packaging to aid a consumer in identifying the product and/or composition so desired. The present invention is also related to methods of merchandising consumer products.

### SUMMARY OF THE INVENTION

According to one aspect of the present invention, an assortment or array of consumer products is provided. In accordance with one of the preferred embodiments, there has now been provided an array of antiperspirant products comprising a first antiperspirant product sold under a first brand name; and a second antiperspirant product also sold under the first brand name, the second antiperspirant product being marketed as clinical strength or prescription strength and being substantially devoid of an antiperspirant active comprising aluminum chloride.

According to another aspect of the present invention, methods of merchandising consumer products are provided. In accordance with one of the preferred embodiments, there has now been provided a method of merchandising antiperspirant products comprising the step of communicating to prospective buyers of antiperspirant products an efficacy comparison between a first antiperspirant product that is marketed as clinical strength or prescription strength and a second antiperspirant product that is not marketed as clinical strength or prescription strength. The first antiperspirant product comprises an antiperspirant active that is substantially devoid of aluminum chloride.

In accordance with another preferred embodiment, there has now been provided a method of merchandising antiperspirant products comprising the step of communicating to prospective buyers of antiperspirant products an underarm skin irritation comparison between a first antiperspirant product that is marketed as clinical strength or prescription strength and a second antiperspirant product that is not marketed as clinical strength or prescription strength. The first antiperspirant product comprises an antiperspirant active that is substantially devoid of aluminum chloride.

In accordance with yet another preferred embodiment, there has now been provided a method of merchandising antiperspirant products comprising the step of communicating to prospective buyers of antiperspirant products a comparison between a first antiperspirant product that is marketed as clinical strength or prescription strength and a second antiperspirant product that is also marketed as clinical strength or prescription strength, wherein the first antiperspirant product comprises an aluminum chloride active and the second antiperspirant product comprises an antiperspirant active that is substantially devoid of aluminum chloride.

### BRIEF DESCRIPTION OF THE DRAWINGS

FIG. 1 is a front elevation view of an exemplary line of antiperspirant products, comprising two products of varying communicated efficacy levels.

FIG. 2 is a front elevation view of a second exemplary line of antiperspirant products, wherein each of the individual products include similar textual indicia but are distinguished from one another by non-textual identifiers, such as by color-for example, one product package being a dark blue color and the adjacent product package being a light blue color.

FIG. 3 is a front elevation view of a third exemplary line of antiperspirant products, wherien the adjacent products comprise synergistic, geometrical product identifiers associated with their packaging to communicate to a consumer that the two products should be bought and/or used in conjunction with each other.

FIG. 4 is a front elevation view of a fourth exemplary line of antiperspirant products, wherein the adjacent products comprise both synergistic, textual product identifiers and synergistic, geometrical product identifiers.

FIG. 5 is a front elevation view of a fifth exemplary line of antiperspirant products, wherein the adjacent products employ synergistic packaging features (i.e., mating features) to communicate to a consumer that the two products should be bought and/or used in conjunction with each other.

FIG. 6 is a front elevation view of a sixth exemplary line of antiperspirant products, wherein the distinguishing product identifiers are included on the respective secondary packaging of the products.

### DETAILED DESCRIPTION OF THE INVENTION

While the specification concludes with the claims particularly pointing and distinctly claiming the invention, it is believed that the present invention will be better understood from the following description.

As used herein, “comprising” means that other steps and/or ingredients can be added. This term encompasses the terms “consisting of” and “consisting essentially of”. The phrase “consisting essentially of” means that the invention may include additional steps and/or ingredients, but only if the additional ingredients and/or steps do not materially alter the basic and novel characteristics of the claimed invention or methods.

All percentages, parts and ratios are based upon the total weight of the topical compositions of the present invention and all measurements made are at 25° C., unless otherwise specified. All such weights as they pertain to listed ingredients are based on the active level and, therefore; do not include carriers or by-products that may be included in commercially available materials, unless otherwise specified.

Embodiments of the present invention involve a line of consumer products having two or more product performance characteristics. The product performance characteristic may include, but is not limited to, the product form, benefit of the product, efficacy level, fragrance level, performance level, and the like. With respect to antiperspirants, the product performance characteristic may include, but is not limited to, the product form, benefit of the product, antiperspirant efficacy, the level of odor protection, fragrance levels, hardness, and/or residue grade. The product line may comprise at least a first product and a second product wherein the two products are differentiated and identified by an “identifier” selected from the group consisting of packaging, color of packaging, shelf marking and mixtures thereof. The identifiers used to differentiate between the sequential products will also differ. Unless stated otherwise, “packaging” includes primary packaging, secondary packaging, and mixtures thereof. Additionally, the products may further include another identifier that is an indicia. As used herein, “indicia” is an identifying marking, which may include words and/or graphics describing a product in use.

The line of consumer products described herein may include a variety of consumer products including but not limited to antiperspirants, deodorants, hair care products, and personal care products including, but not limited to, body wash, body sprays, lotions, creams, and the like. The line of consumer products of the present invention may be from the same manufacturer. A manufacturer is the owner of a particular company, subsidiary and/or brand. Brands may include brand sub lines. By “brand sub line” it is meant a line of products that are of the same type within the same brand and/or within the same manufacturer. For example, the present invention may comprise two antiperspirant products wherein one antiperspirant product is labeled as having a high antiperspirant efficacy such that it is considered to have a significantly higher level of clinically measurable wetness protection than the other antiperspirant product. The antiperspirant product having the label indicating the higher level of antiperspirant efficacy may be considered “prescription strength” while the other antiperspirant composition may be considered regular or non-prescription strength. Since the line of consumer products of the present invention is not limited to only a first and second product, there may be, of course, a variety and number of products that have clinical efficacy levels in between the higher efficacy levels of antiperspirants and the lower efficacy levels of antiperspirants. As used herein, the term “prescription strength” generally refers to an antiperspirant that may have a significantly higher level of clinically measurable wetness protection, when used as directed, than is available in the same product form of a non-prescription strength or regular strength formulation. The term “prescription strength” generally includes other similar labels such as “Dr. recommended”, “clinical strength”, “extra-strength”, “pharmacy grade” and the like. The method for measuring clinical wetness protection is described in U.S. Pat. No. 6,352,688, issued to Scavone et al on Mar. 5, 2002. For example, one embodiment of the present invention may include a first non-prescription strength antiperspirant product wherein the 10-day antiperspirant efficacy is less than about 1.3 and a second prescription strength antiperspirant product that has a 10-day antiperspirant efficacy greater than about 1.3. Or, for example, one embodiment of the present invention may include a first non-prescription strength antiperspirant product wherein the 3-day antiperspirant efficacy is less than about 1.35 and a second prescription strength antiperspirant product that has a 3-day antiperspirant efficacy greater than about 1.35. Of course, one of ordinary skill in the art will also be able to determine that if the efficacy level of one product is greater than the efficacy level of another product, the product with the greater efficacy level may be considered prescription strength.

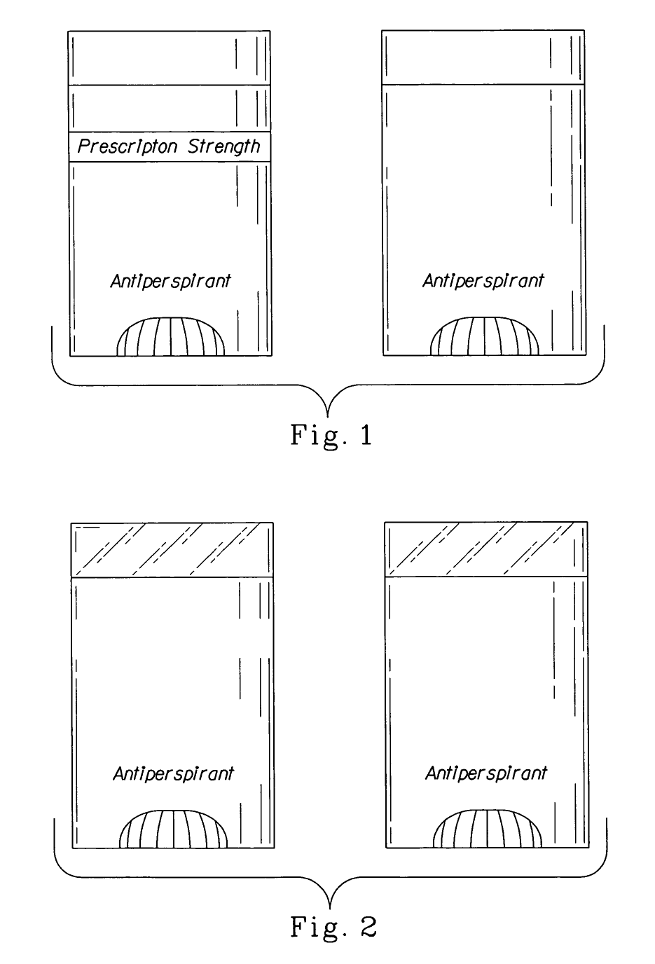

In order to aid a consumer in selecting the right level of antiperspirant protection, each product's identifier may distinguish between a higher level efficacy antiperspirant such as, prescription strength, and a lower level efficacy antiperspirant such as, regular strength (non-prescription strength), such that a consumer will be able to distinguish the products without picking the product up and reading the label. Additionally, if more than two products are used, the product identifier of the additional product or products preferably will be distinguishable from the first, second and any other product identifier within the line. For example, the prescription strength antiperspirant may be packaged differently from the regular strength antiperspirant. Or, for example, the prescription strength antiperspirant may be packaged in a dark blue color while the regular strength antiperspirant may be packaged in a light blue color (see, e.g., FIGS. 2 and 6). A complimentary body spray may also be included within the line wherein the body spray may be packaged in a true blue color. Additionally, an indicia may be included on one product, some products or all products. For example, indicia may be included on the higher efficacy level antiperspirants in the form of a label that indicates that the product is of a higher efficacy level, for example “Prescription Strength” (see, e.g., FIG. 1). These words may be included in one or more worldly languages and may appear on the package in one language or more than one language. Worldly languages may include, but are not limited to, English, Spanish, French, German, Chinese, Japanese, Korean, and Dutch. Or, for example, the indicia may be included on the higher efficacy level antiperspirants in the form of a label that indicates via words and/or graphics “Night” while the lower efficacy level antiperspirant may include a label that indicates via words and/or graphics “Day” to inform, encourage and/or instruct a consumer to follow a particular regimen of use.

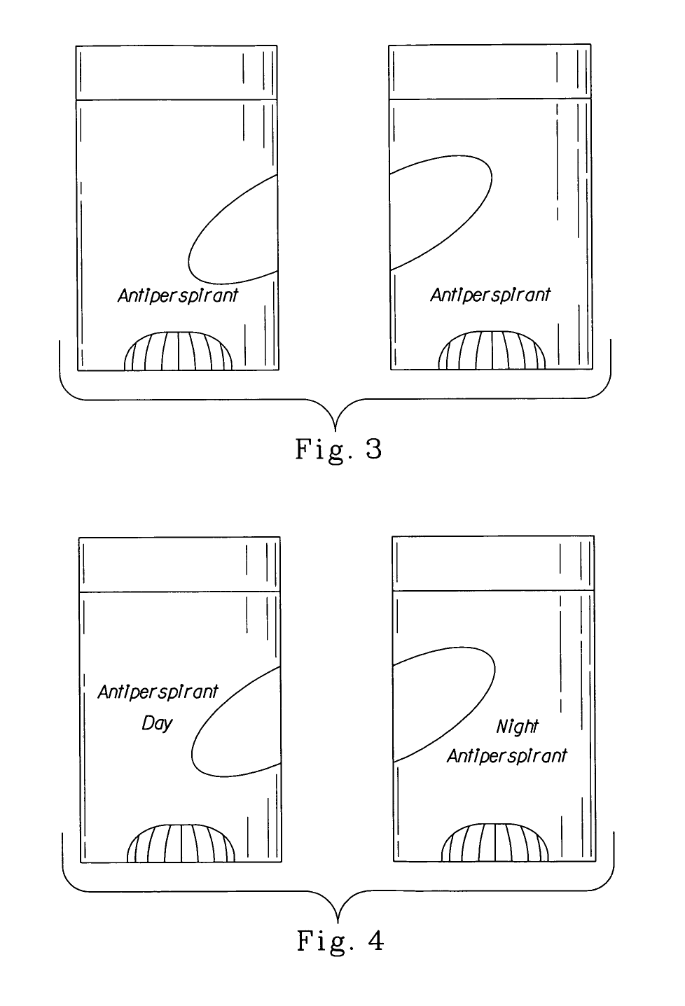

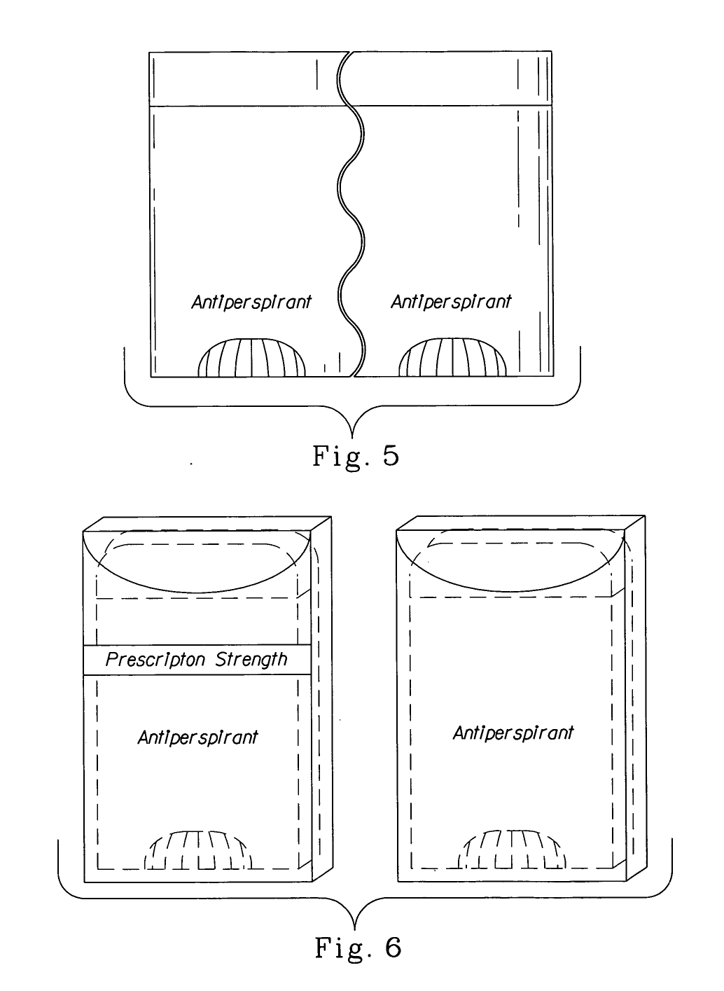

The identifier of the line of consumer products may be synergistic such that when they are displayed or packaged in a unitary form, a consumer knows that the two products should be purchased and/or used in conjunction with each other (see, e.g., FIGS. 3 and 4). The synergistic identifiers together communicate to a consumer that the two or more products of the present invention should be bought and/or used in relation to each other. For example, the first identifier may be a synergistic package that physically interacts with the second identifier that may also be a synergistic package (see, e.g., FIG. 5). Synergistic packaging of the present invention may physically interact by a means selected from the group consisting of interlocking, mating, co-operative assembly and connection. As used herein, “interlocking” describes a connection between two or more products wherein at least two products are locked together, for instance, a tongue and groove. As used herein, “connection” describes a connection between two or more products other than locking the at least two products together, for instance, bonding. As used herein, “mating” or “co-operative assembly” describes the interaction between two or more products wherein at least two products are in simple contact with each other such that the shape of one product conforms to the shape of the other product, for instance, hand in glove or spoon in spoon.

The synergistic identifiers may also comprise indicia wherein two or more indicia together communicate to the consumer that the two or more products should be bought and/or used in relation to each other. The indicia may be labels selected from a group of labels consisting of complimentary labels, continual labels, sequential labels, adjoining labels, and mixtures thereof. By “complimentary labels” it is meant that at least two labels are two halves that make a whole, two colors that are the same, and mixtures thereof. For example, the identifiers may be labels comprising artwork wherein the label of the first product comprises the complimentary half of the label of the second product. For example, the first product may have a label comprising a red, left half of a circle while the second product may have a label comprising a red, right half of a circle which may signal to a consumer that the two products should be bought and/or used in conjunction with each other. By “continual labels” it is meant that the combination of the first and second identifier as a whole creates a complete phrase or a complete picture. Unlike “complimentary labels”, the artwork depicted on continual labels may not be complimentary first and second halves but rather may be a first part of a picture on the first product that continues as a second part of a picture on the second product. For example, a first product may have a label comprising a picture of a lady walking a dog wherein only the lady and the upper part of a leash are seen. The second product may have a label comprising a picture of a bottom half of a leash and the dog that is being walked by the lady on the first identifier. Thus, there is a continuous picture wherein a consumer can identify the two products as those that may be bought and/or used in relation to each other. Or, for example, a first and second identifier as a whole may spell the word “Brand” wherein the first product has a label comprising the letters “Bra” and the second product has a label comprising the letters “nd” wherein a consumer can identify the products spelling a complete word as those that may be bought and/or used in relation to each other. By “sequential labels” it is meant that the products may be displayed communicating the order of use or the time of use. For example, the line of products may comprise a first product having a label comprising the word or number one, the second product having a label comprising the word or number two and a third product having a label comprising the word or number three. Or, for example, there may be a first antiperspirant product with a picture and/or at least one word indicating “Day” and a second antiperspirant product with a picture and/or at least one word indicating “Night”. By “adjoining labels”, it is meant that the first and second identifier may comprise at least one label that adheres to at least two products and indicate to a consumer that the products should be bought and/or used in relation to each other.

It is to be noted that synergistic identifiers of the present invention may not only create a system of complimentary products to be sold, bought or packaged together but it may also encourage consumers to follow a regimen of use. Additionally, the identifiers of the present invention may communicate to the consumer without the additional labor of picking up the product and reading the label.

Method of Use

The line of consumer products of the present invention may be applied topically to the underarm or other suitable area of the skin in an amount effective to reduce or inhibit perspiration wetness. Compositions of the present invention may be applied in an amount ranging from at least about 0.1 gram but no more than about 20 grams, no more than about 10 grams, or no more than about 1 gram.

The line of consumer products of the present invention may be useful as a treatment regimen, for example, treating hyperhidrosis. For example, a first antiperspirant product having a first identifier may be applied to the underarm once while awake and a second antiperspirant product having a second identifier may be applied to the underarm once while asleep. Or, for example, the regular strength antiperspirant product may be applied to the underarm once a day while awake and the prescription strength antiperspirant product may be applied to the underarm once prior to asleep. Of course, it is also suitable if the reverse method is used wherein a person applies the prescription strength while awake and the non-prescription strength prior to going to sleep. Preferably, the application while awake is considered the day, for example, after sunrise to a period prior to sundown while the sleep period is considered the night, for example, sundown to a period prior to sunrise. If this is not the case, the products should be applied according to waking and sleeping periods. Sleeping may include rest for a period of time of at least about 1 hour, at least about 2 hours, at least about 4 hours or at least about 8 hours. Additionally, written and/or graphic instructions may be included to indicate when the respective first and second products should be used.

It is also a desire of the present invention to provide a treatment regimen treating hyperhidrosis by providing a method of convincing a consumer to follow an antiperspirant treatment regimen using only the prescription strength antiperspirant prior to sleeping. Preferably, the prescription strength antiperspirant is not a roll-on; however, any other known or otherwise effective product form for providing topical application of antiperspirant or deodorant active to the desired area of the skin may be used. The prescription strength antiperspirant product of the present invention will also be non-irritating to the skin and may also be absent aluminum chloride.

If aluminum chloride is present, preferably it is in combination with a cyclodextrin inclusion complex (water-releasable encapsulating material). Since aluminum chloride usually limits the selection of fragrances to include within a composition, the use of cyclodextrins with the aluminum chloride allows the use of superior fragrances and aesthetics that would not be available. Thus, the cyclodextrin inclusion complex of the present invention allows for the use of fragrances and/or other acid-liable materials that are not normally deliverable in the presence of aluminum chloride. Suitable cyclodextrins of the present invention may be selected from α-, β-, and γ-cyclodextrins and derivatives thereof. Cyclodextrins of the present invention may be included in soft solid forms as disclosed in by Bacon, et al., U.S. Pat. No. 6,110,449. The cyclodextrins may also be used in other forms as disclosed by Scavone et al., U.S. application Ser. No. 11/132,823, filed May 19, 2005; Scavone et al., U.S. application Ser. No. 11/418,635, filed May 5, 2006; Scavone et al., U.S. application Ser. No. 11/132,804, filed May 19, 2005; and Scavone et al., U.S. application Ser. No. 11/418,607, filed May 5, 2006. Alternative encapsulating materials and technologies may be employed to provide a “reservoir” of releasable materials, such as fragrances. For example, starch-based matrices may be employed to “hold” fragrance materials prior to exposure to bodily-secretions (e.g., perspiration). The encapsulating material may have release mechanisms other than via a solvent; for example, the encapsulating material may be frangible and as such rupture or fracture with applied shear and/or normal forces. The encapsulating material can also include the various capsules and variations thereof, which are disclosed in US Published Patent App No. 2004/0047822.

Actives that may be present as prescription strength antiperspirant products of the present invention may include aluminum zirconium octachlorohydrate, aluminum zirconium octachlorohydrex gly, aluminum zirconium pentachlorohydrate, aluminum zirconium pentachlorohydrex gly, aluminum zirconium tetrachlorohydrate, aluminum zirconium tetrachlorohydrex gly, aluminum zirconium trichlorohydrate, and aluminum zirconium trichlorohydrex gly.

Without being bound by theory, the present inventors have determined that the non-aluminum chloride embodiments of the present invention provide advanced wetness protection to currently prescribed products containing aluminum chloride. The prescription strength embodiments of the present invention, however, are able to provide such benefits without the irritation.

In a clinical study to determine underarm wetness protection, study participants were given two treatment types. The first treatment was the non-aluminum chloride product of the present invention and the second treatment was a commercially available prescribed underarm product. The study participants had the product applied to their underarms every day for seven days wherein a baseline sweat collection occurred on Day 1 prior to product being applied. A sweat collection was then conducted on Day 7. Participants were also monitored for irritation daily during each visit. If a participant had an irritation score of 2 or above, the product application was discontinued and the highest irritation score for that participant product was carried forward for the analysis. The study showed that the commercially available prescribed underarm product was the only product that had irritation scores of 2 or above and required discontinuation. Furthermore, the study demonstrated that the non-aluminum chloride product of the present invention showed a mere 2.6% difference in sweat collection which is not a consumer relevant or noticeable amount. Thus, the results of the study showed the present invention to be as efficacious as the commercially available brand yet superior in overall performance since the participants were left without irritation.

In addition to the present invention being non-irritating, it is also a desire of the present invention to provide a greater selection of perfumes that can be used in the antiperspirant product. Because the non-aluminum product of the present invention eliminates aluminum chloride, a variety of scents can be used that would otherwise have a negative reaction. This is important as the list of scents available may be maximized to appeal to a wider variety of consumers. The non-aluminum chloride product of the present invention may be labeled “prescription strength” to be included in the line of products of the present invention wherein the products may be identified as one prescription strength and one non-prescription strength product. It is also preferred that the non-aluminum chloride product of the present invention be included in a form that is not a roll-on.

If only the prescription strength non-roll antiperspirant of the present invention is used, it is desired that the product is used prior to sleeping. Sleeping may include a period of time of at least about 1 hour, at least about 2 hours, at least about 4 hours or at least about 8 hours. Additionally, written and/or graphic instructions may be included to indicate that the non-roll-on antiperspirant composition is a prescription strength formulation that should be used just prior to a time where a person will be asleep.

Without being limited to the above treatment regimens, the present inventors have found that such treatment regimens may help to treat hyperhidrosis by achieving effective antiperspirant reduction or inhibition over an extended period. Efficacy levels may be evaluated according to the above disclosure. Additionally, testing procedures should be comparable to the recommended method of use. For example, if the treatment regimen instructs one to apply the product at a particular time of day, such as the evening, then the clinical methodology should also reflect this.

Product Form

The antiperspirant compositions of the present invention can be formulated as any known or otherwise effective product form for providing topical application of antiperspirant or deodorant active to the desired area of the skin. Non-limiting examples of such product forms include liquids (e.g., aerosols, pump sprays, roll-ons), solids (e.g., gel solids, invisible solids, wax solid sticks), semi-solids (e.g. creams, soft solids, lotions), and the like. For example, the antiperspirant compositions of the present invention may be semi-solids or solids.

The antiperspirant products are generally stored in and dispensed from a suitable package or applicator device. For example, the package or applicator device may be a cream dispenser with perforated application domes, etc. These packages should be sufficiently closed to prevent excessive loss of volatiles prior to application.

Method of Manufacture

The antiperspirant compositions of the present invention may be prepared by any known or otherwise effective technique, suitable for providing an anhydrous composition of the desired form and having the essential materials described herein. Many such techniques are described in the antiperspirant/deodorant formulation arts for the described product forms.

The line of consumer products of the present invention may be packaged individually in separate containers or they may be packaged together in a unitary form to be sold and bought together. Written and/or graphic instructions may be included in the separate packages or in the unitary packages to instruct a consumer when and how to use the product. Additionally, the product or products may be packaged in a secondary package wherein an outer container embodies the product disclosed therein.

Methods of Merchandising Consumer Products

The present invention is also directed to methods of merchandising consumer products, including, but not limited to, antiperspirant/deodorant products. The methods can be employed to market a single product or an array of products. The methods generally comprise the step of communicating a comparison between at least two different products. Such comparisons can relate to product attributes (negative and/or positive), targeted uses, product “side effects”, and efficacy. For example, comparisons between two different antiperspirant products may relate to efficacy (e.g., wetness protection and/or odor control), skin feel, residue, and/or skin irritation. The information contained in the comparison is unlimited, but preferably (although not required) should include an identification of the consumer care product along with trigger words such, as “compare to” or “compare with” followed by at least a portion of the brand name (or other recognizable identifier) of the comparative product. Comparisons may be made via packaging, advertisements, brochures, product displays, and coupons, for example.

In accordance with one of the preferred embodiments, there has now been provided a method of merchandising antiperspirant products comprising the step of communicating to prospective buyers of antiperspirant products an efficacy comparison between a first antiperspirant product that is marketed as clinical strength or prescription strength and a second antiperspirant product that is not marketed as clinical strength or prescription strength. The first antiperspirant product comprises an antiperspirant active that is substantially devoid of aluminum chloride.

In accordance with another preferred embodiment, there has now been provided a method of merchandising antiperspirant products comprising the step of communicating to prospective buyers of antiperspirant products an underarm skin irritation comparison between a first antiperspirant product that is marketed as clinical strength or prescription strength and a second antiperspirant product that is not marketed as clinical strength or prescription strength. The first antiperspirant product comprises an antiperspirant active that is substantially devoid of aluminum chloride.

In accordance with yet another preferred embodiment, there has now been provided a method of merchandising antiperspirant products comprising the step of communicating to prospective buyers of antiperspirant products a comparison between a first antiperspirant product that is marketed as clinical strength or prescription strength and a second antiperspirant product that is also marketed as clinical strength or prescription strength, wherein the first antiperspirant product comprises an aluminum chloride active and the second antiperspirant product comprises an antiperspirant active that is substantially devoid of aluminum chloride.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 nm” is intended to mean “about 40 mm”.

All documents cited in the Detailed Description of the Invention are, are, in relevant part, incorporated herein by reference; the citation of any document is not to be construed as an admission that it is prior art with respect to the present invention. To the extent that any meaning or definition of a term in this document conflicts with any meaning or definition of the term in a document incorporated herein by reference, the meaning or definition assigned to the term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of antiperspirant products, comprising:
a first antiperspirant product sold under a first brand name;
a second antiperspirant product also sold under the first brand name, the second antiperspirant product being marketed as clinical strength or prescription strength and being substantially devoid of an antiperspirant active comprising aluminum chloride.

2. The array of products according to claim 1, wherien the second antiperspirant product comprises an encapsulated fragrance material.

3. The array of products according to claim 2, wherein the second antiperspirant product is anhydrous and the encapsulated fragrance material comprises water-releasable encapsulating material.

4. The array of products according to claim 1, wherein the second antiperspirant product is not a roll-on product.

5. A method of merchandising antiperspirant products, comprising the step of communicating to prospective buyers of antiperspirant products an efficacy comparison between a first antiperspirant product that is marketed as clinical strength or prescription strength and a second antiperspirant product that is not marketed as clinical strength or prescription strength,
wherein the first antiperspirant product comprises an antiperspirant active that is substantially devoid of aluminum chloride.

6. The method of claim 5, wherein the efficacy comparison relates to wetness protection.

7. The method of claim 5, wherein the efficacy comparison relates to odor mitigation.

8. The method of claim 5, wherein the step of communicating is accomplished with a textual comparison included on packaging of one of the first or second antiperspirant products.

9. The method of claim 5, wherien the step of communicating is accomplished via an advertisement.

10. The method of claim 5, wherein the step of communicating comprises the second antiperspirant product identifying the first antiperspirant product by at least a portion of its brand name.

11. The method of claim 10, wherein the step of communicating further comprises the second antiperspirant product communicating a phrase comprising “compare to” or “compare with.”

12. The method of claim 5, wherein each of the first antiperspirant product and the second antiperspirant product includes instructions for applying the product at night and/or before sleeping.

13. The method of claim 5, wherien the first antiperspirant product comprises an encapsulated fragrance material.

14. The method of claim 5, wherein the first antiperspirant products is not a roll-on product.

15. A method of merchandising antiperspirant products, comprising the step of communicating to prospective buyers of antiperspirant products an underarm skin irritation comparison between a first antiperspirant product that is marketed as clinical strength or prescription strength and a second antiperspirant product that is not marketed as clinical strength or prescription strength,
wherein the first antiperspirant product comprises an antiperspirant active that is substantially devoid of aluminum chloride.

16. The method of claim 15, wherien the first antiperspirant product comprises an encapsulated fragrance material.

17. The method of claim 15, wherein the first antiperspirant products is not a roll-on product.

18. A method of merchandising antiperspirant products, comprising the step of communicating to prospective buyers of antiperspirant products a comparison between a first antiperspirant product that is marketed as clinical strength or prescription strength and a second antiperspirant product that is also marketed as clinical strength or prescription strength, wherein the first antiperspirant product comprises an aluminum chloride active and the second antiperspirant product comprises an antiperspirant active that is substantially devoid of aluminum chloride.

19. The method of claim 18, wherein the comparison relates to wetness protection.

20. The method of claim 18, wherein the comparison relates to underarm skin irritation.
